(19) United States
(12) Patent Application Publication
(10) Pub. No.: US 2008/0206165 A1
(43) Pub. Date: Aug. 28, 2008

(54) Single-use personal care products and kits comprising same

(76) Inventors: Shekhar Mitra, Indian Hill, OH (US); David Matthew Groh, Lebanon, OH (US); Gordon Gerald Guay, Chelmsford, MA (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 12/001,331

(22) Filed: Dec. 11, 2007

Related U.S. Application Data
(60) Provisional application No. 60/874,086, filed on Dec. 11, 2006.

(51) Int. Cl. A61K 8/18; A61K 8/72; A61K 8/00; A61Q 15/00; A61Q 11/00; A61Q 9/02

(52) U.S. Cl. 424/49; 424/73; 424/70.11; 424/65; 424/70.1

(57) ABSTRACT

Single-use personal care products and kits comprising the same are disclosed. At least some of the single-use products include a pre-measured amount of a shaving composition.

This application claims the benefit of U.S. Provisional Application No. 60/874,086 filed Dec. 11, 2006.

### FIELD OF THE INVENTION

The present invention relates to single-use personal care products and kits comprising the same. At least some of the single-use products include a pre-measured amount of a shaving composition.

### BACKGROUND OF THE INVENTION

Currently, a widely used form of shaving preparation is the type referred to as a post-foaming shave gel. These post-foaming shave gels are now well-known and have been described, for example, in U.S. Pat. No. 2,995,521 (Bluard), U.S. Pat. No. 3,541,581 (Monson), U.S. Pat. No. 4,405,489 (Sisbarro), U.S. Pat. No. 4,528,111 (Su), U.S. Pat. No. 4,651,503 (Anderson), U.S. Pat. No. 5,248,495 (Patterson), U.S. Pat. No. 5,308,643 (Osipow), U.S. Pat. No. 5,326,556 (Barnet), and U.S. Pat. No. 5,500,211 (George). Such compositions generally take the form of an oil-in-water emulsion in which the post-foaming agent, generally a volatile (i.e., low boiling point) aliphatic hydrocarbon, is solubilized in the oil phase, and the water phase comprises a water-dispersible soap or interrupted soap component. The product is generally packaged in an aerosol container with a barrier, such as a piston or collapsible bag, to separate the post-foaming gel from the propellant required for expulsion of the product. The product is dispensed as a clear, translucent or opaque gel that is substantially free from foaming until it is spread over the skin, at which time it produces a foam lather generated by the volatilization of the volatile hydrocarbon foaming agent.

The method of common dispensing of post foaming shaving compositions, via an aerosol container, has a number of consumption negatives. As the amount of shaving composition dispensed is predicated upon the user and their ability to dispense the proper amount, many times an excess amount of the shaving composition is dispensed. In addition, many aerosol dispensers continue to “leak” or dispense the composition in small amounts after the user has released the dispensing button on the aerosol container. Lastly, the shaving composition must be spread by a consumer's hand around the area to be shaved, leaving excess shaving composition on the consumer's hand.

Accordingly, the need remains for a shaving composition and a container/dispenser which dispenses a pre-measured dosage of a shaving composition and facilitates application to the consumer's skin while eliminating many of the common negatives associated with aerosol and packaging today.

### BRIEF DESCRIPTION OF THE DRAWINGS

While the specification concludes with claims particularly pointing out and distinctly claiming the subject matter which is regarded as forming the present invention, it is believed that illustrative embodiments of the present invention may be better understood from the following description taken in conjunction with the accompanying drawings, in which:

FIG. 1 is a perspective view of an exemplary single-use product of the present invention constructed from flexible sheet material;

FIG. 2 is a cross-sectional view of the product shown in FIG. 1 and taken through line II-II;

FIG. 3 is a perspective view of another exemplary single-use product of the present invention constructed from tubular members;

FIG. 4 is a perspective view of the product shown in FIG. 3 with its cap/seal removed to expose an expanded applicator;

FIGS. 5 and 6 are respective elevation and perspective views of a third exemplary single-use product of the present invention;

FIG. 7 is a perspective view of an exemplary single-use product of the present invention comprising multiple compartments; and

FIG. 8 is a cross-sectional view of another exemplary single-use product of the present invention comprising multiple compartments.

### DETAILED DESCRIPTION OF EXEMPLARY EMBODIMENTS AND FEATURES

The single-use apparatus employed in the present invention is one designed for dispensing and application of pre-measured amounts of personal care compositions. A representative, non-limiting list of personal care compositions that may be dispensed/applied by the single-use apparatuses of the present invention, includes shaving compositions, skin care compositions, hair care compositions, antiperspirant/deodorant compositions, hair growth management compositions (both inhibitors and stimulants), oral care compositions, personal cleansing compositions, cosmetics, fragrances, acne compositions, sunscreen compositions, and baby care compositions.

In accordance with one aspect of the present invention, the personal care composition is a shaving composition. Exemplary embodiments can permit application of a suitable amount of a shaving composition to the skin of a consumer without the consumer coming into direct contact with the composition and without the associated guess work by the consumer to dispense the proper amount. Such exemplary embodiments may also provide increased hygiene benefits due to its designed use, as well as a more streamlined shaving process by substantially decreasing the level of and/or eliminating hand washing steps in the shaving process.

Exemplary single-use products of the present invention generally comprise a dispenser including one or more compartments, an applicator having a skin-contacting surface, and a single-use volume of a personal care composition contained within the compartment(s). The dispensers preferably employ a seal and/or valve to inhibit/prohibit premature dispensing of the personal care composition. When a seal is employed, the placement and type of the seal is unlimited-illustrative seals and their respective placements are shown in the figures included herein. One of ordinary skill in the art will recognize that the single-use products of the present invention may encompass a number of different dispenser embodiments, including, but not limited to those disclosed in U.S. Pat. Nos. 6,902,335 and 6,007,264.

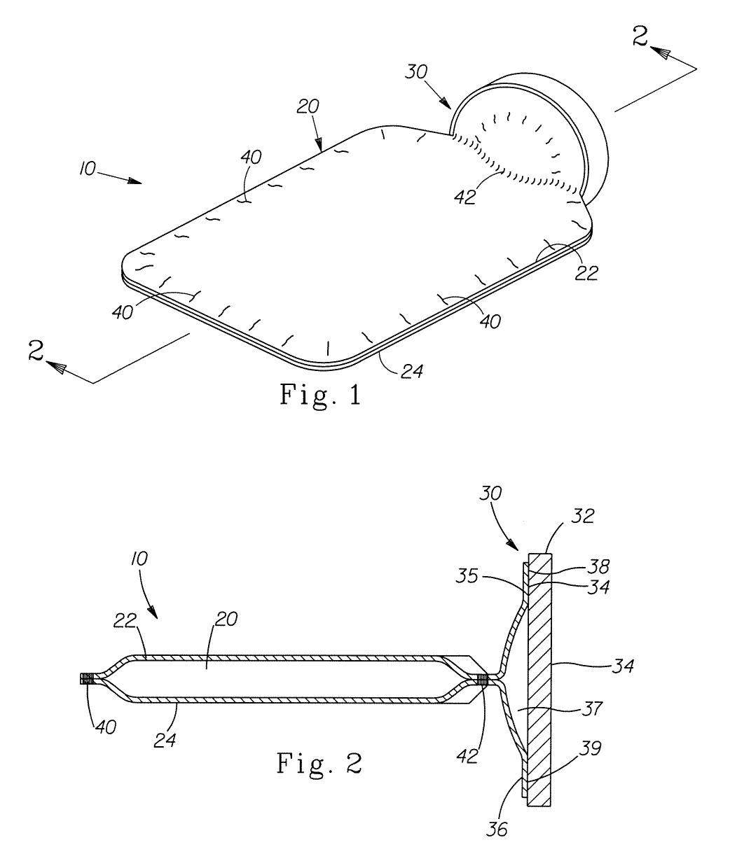

Exemplary single-use products will now be described with reference to the figures. Referring generally to FIGS. 1 and 2, a dispenser 10 is shown including a compartment 20 defined by opposing layers 22 and 24 of a substantially flexible material, and an applicator 30. Dispenser 10 is also shown having seals 40 and 42 to define a closed compartment 20 prior to use of dispenser 10. Seal 42 is designed to be a frangible seal that is rupturable when a user applies pressure to compartment 20, which in turn permits the flow of a personal care composition from compartment 20 to applicator 30. Thus, seal 42 is configured to have a lower rupture pressure than seal 40, so that a contained personal care composition is channeled in the direction towards applicator 30 and not out of the sides or back of compartment 20. Alternative embodiments of the present invention may employ a valve (e.g, a flap valve) rather than a frangible seal to inhibit flow from compartment 20 to applicator 30 prior to use. Alternative embodiments may also employ a removable seal that covers a portion (or all) of applicator 30, whereby a user partially or completely peels the removable seal sufficiently away from skin-contacting surface 32 to permit dispensing of a personal care composition contained within compartment 20. Of course, one of ordinary skill in the art will recognize the entire dispenser may be enclosed in suitable packaging to maintain cleanliness or even maintaining a sterile environment. One of ordinary skill will also recognize that there is no particular size restriction placed upon the single-use products of the present invention or the amount of personal care composition dispensed other than general considerations of size, weight and resultant ease of use.

Exemplary applicator 30 includes a porous substrate or pad 32 having a skin-contacting surface 34, optional wings 35 and 36, and an optional expansible chamber 37. By way of example only, and as shown in FIGS. 1 and 2, wings 35 and 36 are defined by extensions of opposing layers 22 and 24. They may alternatively be separately formed and attached to compartment 20. Pad 32 is attached, via an adhesive for example, to wings 35 and 36 at locations 38 and 39. Wings 35 and 36 may help to support porous pad 32 depending on its mechanical properties, and can also help create at least a partial barrier between a user's fingers and the personal care composition being dispensed. Pad 32 is shown as extending radially beyond wings 35 and 36; however, pad 32 may be co-extensive with wings 35 and 36, or the wings may extend beyond pad 32. In an alternative embodiment, wings 35 and 36 may serve as a skin-contacting surface, with pad 32 being eliminated.

With the presence of optional chamber 37, the personal care composition flows from compartment 20 into chamber 37 upon rupturing frangible seal 42. The expansion of the chamber walls and the resilience of the applicator pad 32 allow the composition to spread out behind and into the applicator pad for application to the skin.

Opposing layers 22 and 24 can be made from a variety of generally flexible materials, including, for example, polymeric films, metallic films (foil), nonwovens, wovens, fabrics, coated papers (e.g., wax-coated paper) and combinations thereof. The layers of materials employed may be single or multi layered structures, and may be transparent, translucent and/or opaque. In one preferred embodiment, opposing layers 22 and 24 comprise laminated foils, such as, for example, polyester and aluminum sheet stocks laminated together via adhesives and available from the Pechiney Corporation. Opposing layers 22 and 24 may be made from a single sheet stock with the sheet stock being folded to define compartment 20, or may be made from separate sheet stocks that are affixed to one another. Instead of being manufactured from sheet stock materials, as shown and described thus far, compartment 20 may be made as a thermoformed or molded container. The wall thickness and material can be chosen to permit a user to create pressure on the personal care composition contained therein. By way of example only, compartment 20 may be defined as polyolefin blow-molded container.

One of ordinary skill in the art will recognize that substrate or pad 32 may be constructed from various materials. A representative, non-limiting list of suitable materials includes natural and synthetic foams, nonwoven webs, woven webs, felts, flocked fiber substrates, apertured films, and combinations thereof. The substrate or pad preferably has good reservoiring and wicking characteristics for the proper release and spreading of the composition to the skin. For application of shaving compositions, the substrate or pad is preferably designed and configured for generating lather. The substrate or pad may be constructed into any suitable shape including, but not limited to, square, rectangle, oval, circular and various novelty shapes. Even where the substrate or pad 32 is made from porous materials, additional channels or conduits may be formed in the material to create particular flow directions and flow rates.

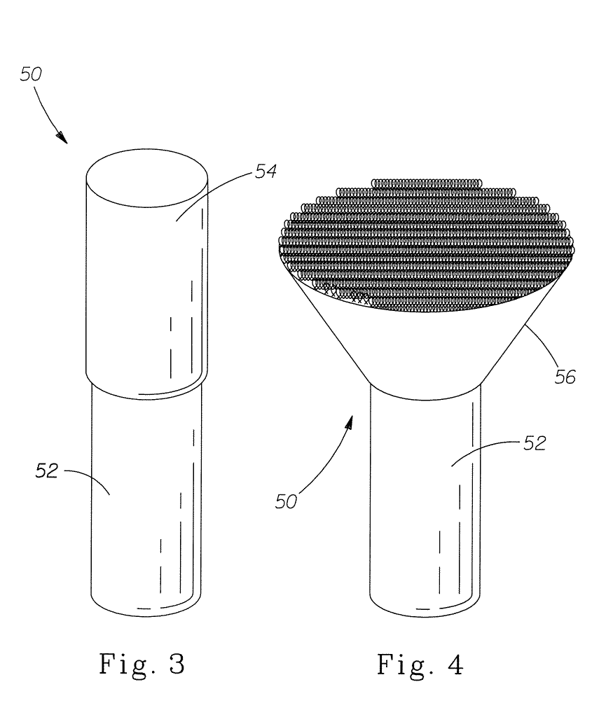

Single-use dispensers of the present invention may employ other constructive aspects, including manufacturing the respective personal care compartments by thermal forming techniques rather than constructing them from one or more sheet materials. For example, and with reference to FIG. 3, dispenser 50 comprises a tubular body 52 and removable tubular cap 54. Tubular body 52 defines a compartment for containing a single-use volume of a personal care composition. Tubular cap 54 is removed by a consumer to expose an applicator 56, as can be seen in FIG. 4. Applicator 56 may comprise compressible foam, for example, that expands upon removing tubular cap 54. Other materials may also be employed for applicator 56. Tubular body 52 may be flexible so that a user can compress the same to urge the contained personal care composition into applicator 56. Tubular body 52 may alternatively be fairly rigid, whereby a user can invert the dispenser so that gravity facilitates transfer of the contained personal care composition into applicator 56. Although tubular body 52 and tubular cap 54 are illustrated as two separate components, they may also be formed as a single component with a means of separating a portion of the same as an added design element (for example a line of weakness formed in the tubular element). Tubular body 52 and tubular cap 54 may be made by numerous techniques including, for example, injection molding, extrusion, thermoforming, and blow molding.

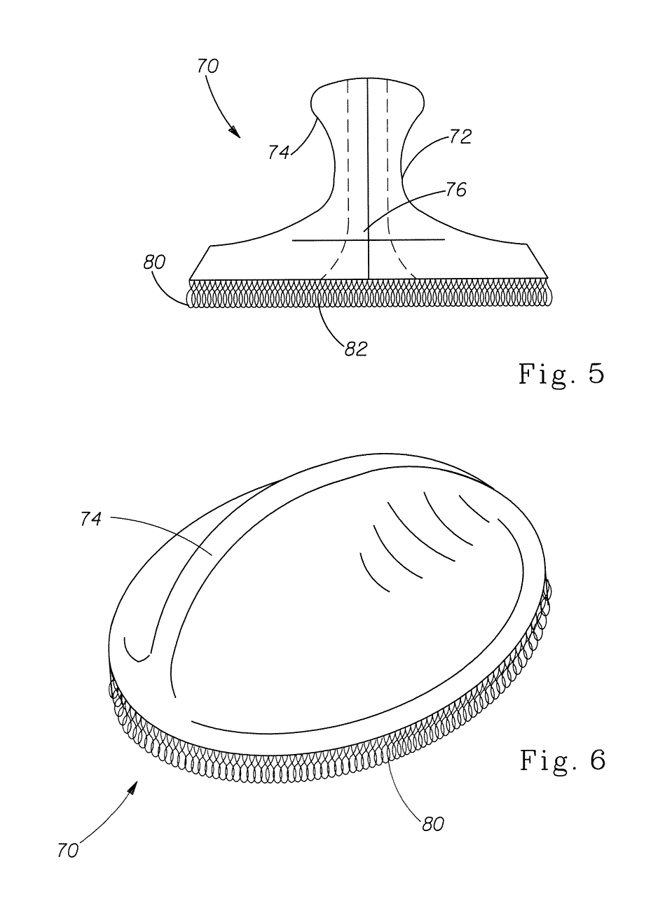

FIGS. 5 and 6 show another exemplary dispenser 70 comprising a body 72 that defines both an ergonomic handle 74 and compartment or reservoir 76 for containing a personal care composition. Dispenser 70 also comprises an applicator 80 and removable seal 82.

Single-use dispenser embodiments of the present invention may employ a separate applicator (not shown) that is capable of engaging a single-use compartment, chamber or other disposable container to dispense and apply a contained personal care composition. For example, dispenser 10 shown in FIGS. 1 and 2 could comprise a compartment 20 that is closed prior to use by seals 40 and 42, and a separate applicator that can be temporarily connected to compartment 20 (the applicator may also be originally connected to compartment 20 and then subsequently removed for attachment to another compartment). The separate applicator may comprise a stem or other projection configured for piercing seal 42 and/or inserting into compartment 20, with the stem or projection employing a channel to transfer a contained personal care composition from compartment 20 to a skin-contacting surface of the separate applicator. Multiple single-use compartments can be bundled or otherwise sold with a separate applicator for multiple uses. Personal care compositions contained within the single-use compartments may be similar, or dissimilar so as to a plurality of experiences and/or benefits. For example, a kit may comprise a separate applicator along with three distinct compartments: a first compartment containing a shaving composition, a second compartment containing an after shave composition, and a third compartment containing a moisturizing composition.

Shaving compositions are one of the preferred personal care compositions to be dispensed and applied using the dispensers described herein. Exemplary shaving compositions comprise, in percent by weight, from about 60% to about 93% of water, from about 2% to about 25% of a water dispersible (or soluble) surface active agent, from about 0.005% to about 2% of a lubricious water soluble polymer, from about 0.0005% to about 3% of a hydrogel-forming polymer, and optionally from about 1% to about 6% of a volatile post-foaming agent. Each of these components is described more fully below.

The water dispersible surface active agent is preferably one that is capable of forming a lather and may comprise a soap, an interrupted soap, a detergent, an anionic surfactant, a non-ionic surfactant or a mixture of one or more of these. The soaps may include, for example, the sodium, potassium and lower alkanolamine (preferably triethanolamine) salts of C12 22, preferably C14 18, fatty acids. Typical fatty acids include lauric, myristic, palmitic and stearic acid and mixtures thereof. The preferred fatty acids are palmitic and stearic. The interrupted soaps may include, for example, the sodium, potassium and lower alkanolamine (preferably triethanolamine) salts of N-fatty acyl sarcosines, wherein the fatty acyl moiety has 12 to 22, preferably 14 to 18, carbon atoms. Typical sarcosines include stearoyl sarcosine, myristoyl sarcosine, palmitoyl sarcosine, oleoyl sarcosine, lauroyl sarcosine, cocoyl sarcosine and mixtures thereof. The soaps and the interrupted soaps may be utilized in the preneutralized form (i.e., as the sodium, potassium or alkanolamine salt) or in the free acid form followed by subsequent neutralization with sodium hydroxide, potassium hydroxide and/or lower alkanolamine (preferably triethanolamine). In any event, the final shaving composition preferably contains sufficient base to neutralize or partially neutralize the soap component and adjust the pH to the desired level (typically between 5 and 10, more typically between 6 and 9). It is most preferred that the shaving composition includes a soap (e.g., triethanolamine palmitate/stearate), an interrupted soap (e.g., triethanolamine stearoyl/myristoyl sarcosinate), or a mixture thereof.

The water dispersible surface active agent may also optionally include a non-ionic, amphoteric and/or anionic surfactant. Suitable non-ionic surfactants will typically have an HLB of 9 or more and include the polyoxyethylene ethers of fatty alcohols, acids and amides, particularly those having 10 to 20, preferably 12 to 18, carbon atoms in the fatty moiety and about 2 to 60, preferably 4 to 30, ethylene oxide units. These include, for example, Oleth 20, Steareth 21, Ceteth 20, Laureth 4 and Laureth 23. Other non-ionic surfactants include the polyoxyethylene ethers of alkyl substituted phenols, such as Nonoxynol-4 and Nonoxynol-20, fatty alkanolamides such as Lauraminde DEA and Cocamide MEA, polyethoxylated sorbitan esters of fatty acids, such as Polysorbate 20, lauryl polyglucoside, sucrose laurate, and polyglycerol 8 oleate. Suitable amphoteric surfactants include, for example, the betaines and sultaines such as cocoamidopropyl betaine, coco dimethyl carboxymethyl betaine, coco sultaine and the like. Suitable anionic surfactants include, for example, the sodium, potassium, ammonium and substituted ammonium salts (such as the mono-, di- and triethanolamine salts) of C8 C22, preferably C12 C18, alkyl sulfates (e.g., sodium lauryl sulfate, ammonium lauryl sulfate), alkyl sulfonates (e.g., ammonium lauryl sulfonate), alkylbenzene sulfonates (e.g., ammonium xylene sulfonate), acyl isethionates (e.g., sodium cocoyl isethionate), acyl lactylates (e.g., sodium cocoyl lactylate) and alkyl ether sulfates (e.g., ammonium laureth sulfate). The surface active agent may typically include up to about 10%, preferably 1 to 8%, of non-ionic, amphoteric and/or anionic surfactants.

Exemplary lubricious water soluble polymers will generally have a molecular weight greater between about 300,000 and 15,000,000 daltons, preferably more than about one million daltons, and will include a sufficient number of hydrophilic moieties or substituents on the polymer chain to render the polymer water soluble. The polymer may be a homopolymer, copolymer or terpolymer. Examples of suitable lubricious water soluble polymers include polyethylene oxide, polyvinylpyrrolidone, and polyacrylamide. A preferred lubricious water soluble polymer comprises polyethylene oxide, and more particularly a polyethylene oxide with a molecular weight of about 1 to about 5 million daltons. Particularly suitable polyethylene oxides include, for example, PEG 23M (MW 1 million), PEG 45M (MW≈2 million) and PEG 90M (MW≈4 million).

The hydrogel-forming polymer may be a highly hydrophilic polymer that, in water, forms organized three-dimensional domains of approximately nanometer scale. The hydrogel-forming polymer generally has a molecular weight greater than about one million daltons (although lower molecular weights are possible) and typically is at least partially or lightly crosslinked and may be at least partially water insoluble, but it also includes a sufficient number of hydrophilic moieties so as to enable the polymer to trap or bind a substantial amount of water within the polymer matrix and thereby form three-dimensional domains. It has been found that shave gel compositions that include the hydrogel-forming polymer have improved gel structure and reduced coefficient of friction (i.e., increased lubricity). Examples of suitable hydrogel-forming polymers include a polyacrylic acid or polymethacrylic acid partially esterified with a polyhydric alcohol, hydrophilic polyurethanes, lightly crosslinked polyethylene oxide, lightly crosslinked polyvinyl alcohol, lightly crosslinked polyacrylamide, hydrophobically modified hydroxyalkyl cellulose, hydroxyethyl methacrylate, and crosslinked hyaluronic acid.

An exemplary hydrogel-forming polymer comprises polyacrylic acid partially esterified (e.g., about 40% to 60%, preferably about 50%, esterified) with glycerin. Such a polymer includes glyceryl acrylate/acrylic acid copolymer (MW>one million). It is believed that the glyceryl acrylate/acrylic acid copolymer forms a clathrate that holds water, which, upon release supplies lubrication and moisturization to the skin. A preferred source of glyceryl acrylate/acrylic acid copolymer is available from ISP Technologies, Inc. (United Guardian Inc.) under the tradename Lubrajelg, particular the form known as Lubrajel® oil which contains about 1.0%1.3% glyceryl acrylate/acrylic acid copolymer in aqueous glycerin (˜40% glycerin). Lubrajel® oil also includes about 0.6% PVM/MA copolymer (also known as methoxyethylene/maleic anhydride copolymer), which may further contribute to the lubricity of this source.

The optional post-foaming agent may be any volatile hydrocarbon or halohydrocarbon with a sufficiently low boiling point such that it will volatilize and foam the gel upon application to the skin, but not so low that it causes the gel to foam prematurely. The typical boiling point of such an agent generally falls within the range of 20° C. to 40° C. Preferred post-foaming agents are selected from saturated aliphatic hydrocarbons having 4 to 6 carbon atoms, such as n pentane, isopentane, neopentane, n butane, isobutane, and mixtures thereof. Most preferred is a mixture of isopentane and isobutane in a weight ratio (IP:IB) of about 1:1 to about 9:1, preferably about 2:1 to about 7:1, most preferably about 3:1. The post-foaming agent will normally be selected so as to provide a vapor pressure at 20° C. of about 3 to about 20 psig, and preferably from about 5 to about 15 psig. The post-foaming agent will be present in an amount to provide the shaving composition with a sufficiently rapid turnover that is, transition from gel to foam when contacted with the skin typically, in about 2 to about 30 seconds, preferably in about 5 to about 15 seconds.

Although not necessary to forming a useful shaving composition, other cosmetic ingredients may be advantageously added to improve the application aesthetics and/or achieve other shave benefits. For example, the shaving composition may include one or more of the following components: wetting agents, skin conditioning agents (e.g., vitamins A, C and E, aloe, allantoin, panthenol, alpha-hydroxy acids, phospholipids, triglycerides, botanical oils, amino acids), foam boosters, emollients, humectants (e.g., glycerin, sorbitol, propylene glycol), fragrances, colorants, antioxidants, preservatives, etc.

It may be advantageous to include a sorbitan fatty ester or a sucrose fatty ester, typically in an amount of about 0.1% to about 3%, preferably about 0.3% to about 2%, by weight. These materials have multifunctional properties of emulsifier, moisturizer and anti-irritant. Sorbitan fatty esters include sorbitan stearate, sorbitan oleate, sorbitan isostearate, sorbitan laurate, sorbitan dioleate, etc. Sucrose fatty esters include sucrose stearate, sucrose oleate, sucrose isostearate, sucrose cocoate, sucrose distearate, etc. The sorbitan esters and sucrose esters may be mixtures of mono-, di- and tri-esters.

It may also be desirable to include an ester of a fatty acid, typically in an amount of about 0.5% to about 5%, preferably about 1% to about 4%, by weight. Useful fatty esters include glyceryl fatty esters such as, for example, glyceryl oleate and glyceryl dioleate, and fatty alcohol esters such as, for example, isostearyl linoleate, isocetyl oleate, and isostearyl isostearate. These materials provide emolliency, lubrication and gel structure.

It may further be desirable to include a propoxylated fatty amide, typically in an amount of about 0.5% to about 5%, preferably about 1% to about 3%, by weight. The propoxylated fatty amide will typically have from 1 to 3 propoxyl groups attached to a hydroxyloweralkyl fatty amide. Thus, suitable propoxylated fatty amides include, for example, PPG 2-hydroxyethyl coco/isostearamide, PPG 3-hydroxyethyl linoleamide, and PPG 2-hydroxyethyl cocamide.

The shaving composition may include a water-soluble gelling aid or thickening agent to improve its consistency and stability, as well as to adjust its viscosity. These may include, for example, hydroxyalkyl cellulose polymers such as hydroxyethyl cellulose and hydroxypropyl cellulose (sold under the trademarks “Natrosol” and “Klucel” respectively), PEG-150 distearate, carboxymethyl cellulose, and cellulose methyl ether (sold under the trademark “Methocel”). Other suitable materials include the polysaccharide gums such as, for example, xanthan gum, carrageenan gum, guar gum, locust bean gum, and hydroxypropyl guar gum.

The amount, formulation and type of shaving composition disposed within the single-use dispensers varies generally for example with the body-part to be shaved, the shaving environment, skin type, sensitivity level, and the user marketed to. In accordance with one preferred embodiment, the volume of shaving composition contained within the dispenser compartment ranges from about 0.5 ml to about 30 ml. It should be understood, that smaller and larger volumes are also contemplated by the present invention.

Applicators of the single-use dispensers may be loaded or impregnated (by the manufacturer and/or consumer) with a personal care composition that is different than that contained in the dispenser compartment. The personal care compositions may be designed to provide two distinct benefits and/or experiences, or may be designed to provide a single benefit that is “activated” or “primed” when the two compositions contact one another (for example, an exothermic reaction occurs upon mixing the two compositions to create heat). Accordingly, a user may apply the two different personal care compositions sequentially, or alternatively apply a combined/mixed composition that comprises the two different compositions (the mixing can occur before and/or during application to one's body).

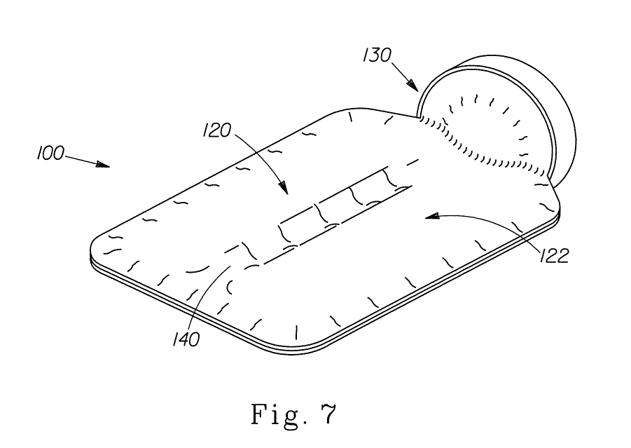

Single-use products of the present invention can also include dispensers having multiple compartments for delivering multiple (similar or dissimilar) compositions or composition components. As can be seen in FIG. 7, exemplary dispenser 100 includes compartments 120 and 122 separated by a longitudinally extending seal 140. Dispenser 100 has a single applicator 130 for dispensing and applying compositions or composition components contained in both of compartments 120 and 122. In accordance with one embodiment, seal 140 is designed as a permanent seal, so that the individual compositions or composition components are not pre-mixed prior to engaging applicator 130.

In an alternative embodiment, seal 140 is a frangible seal that can be ruptured to permit pre-mixing of the materials contained within compartments 120 and 122. By way of example, compartment 120 may contain a first shave gel base and an oxidizing agent, and compartment 122 contain a second shave gel base and a reducing agent. The first shave gel base and the second shave gel base may each independently include an oil-in-water emulsion including, by weight, about 55% to about 90% water, about 3% to about 20% of a water-dispersible surface active agent capable of forming a lather, and optionally about 1% to about 6% of a volatile self-foaming agent. The oxidizing agent and the reducing agent are selected and are present in such proportion to provide an exothermic reaction upon mixing of the oxidant component and the reductant component prior to and/or during use of the shaving composition.

Suitable oxidizing agents may include peroxides, e.g., hydrogen peroxide, benzoylperoxide, peroxomonosulfate, peroxodisulfate, urea hydrogen peroxide, and t-butyl peroxide. Suitable reducing agents are those that will react with the oxidizing agent when the two components of the formulation are mixed, to generate an exothermic reaction. Suitable reducing agents should also be safe for use on human skin in the amounts used in the formulation. The reducing agent may include, for example, thiosulfate and sulfite compounds, such as sodium sulfite, sodium thiosulfate, ammonium thiosulfate, potassium thiosulfate, and thiourea. Other suitable reducing agents include compounds with a thiourea backbone, such as 1,5 diethyl-2-thiobarbituric acid or its derivatives, or ascorbic acid. Mixtures of the above reducing agents, and other suitable reducing agents, may also be used. One or both of the shave gel bases may include a catalyst selected to catalyze the exothermic reaction between the oxidizing agent and the reducing agent, and/or a neutralizing agent selected to neutralize acid generated by the exothermic reaction between the oxidizing agent and the reducing agent. Self-heating shaving compositions can also be contained and dispensed from an embodiment without an intermediate frangible seal, wherein the mixing of the two shave gel bases occurs during and after engagement with the dispenser applicator.

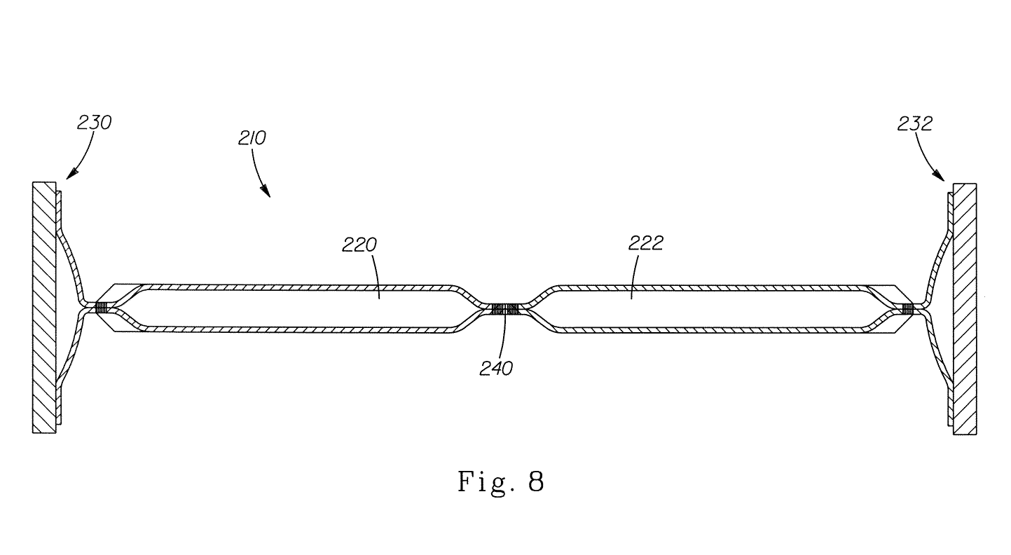

Other multi-compartment dispensers may contain dedicated applicators for each of the individual compartments. For example, a cross-sectional view of a multi-compartment dispenser 210 is shown in FIG. 8, comprising a first compartment 220 with its associated applicator 230, a second compartment 222 with its associated applicator 232, and a seal 240 disposed between the two compartments. Dispenser 210 may include other features as described above. Although FIG. 8 illustrates the two compartments and their respective applicators as being essentially the same in all regards, multi-compartment dispensers of the present invention are not so limited. The size, geometry, material make-up, and color of the compartments and/or applicators may differ from one another. Furthermore, the volume of the personal care composition in first compartment 220 may be the same or different than the volume of the personal care composition in second compartment 222. The skilled artisan would readily appreciate that more than two compartments may also be employed.

In one of the preferred embodiments, both of compartments 220 and 222 contain a shaving composition. The individual compositions may be the same or may be physically or chemically different from each other. A user may be instructed to use the shaving composition disposed within compartment 220 to shave a first body part, and the shaving composition disposed within compartment 222 to shave a second body part. The respective body parts may include each leg, the bikini area, the underarm, and the face.

In another preferred embodiment, compartment 220 contains a shaving composition, while compartment 222 contains a different type of personal care composition that is not intended to be used for shaving. For example, compartment 222 may contain a body wash, a moisturizer, a shampoo, a hair conditioner, a hair styling composition, an antiperspirant composition, a deodorant composition, a body spray, an aftershave composition, or a toothpaste. That is, single-use products of the present invention may contain a shaving composition and one or more personal care compositions that are typically applied within a relatively short period (e.g., within 1 hour) of shaving one's body. Some particularly preferred combinations for males include a shaving composition paired with an aftershave composition or an antiperspirant/deodorant composition. Some particularly preferred combinations for females include a shaving composition paired with a body wash, moisturizer, shampoo, or hair conditioner.

The present invention is also directed to shaving-related kits, including: 1) a plurality of single-use products comprising shaving compositions as described above, and 2) a disposable razor or a plurality of replacement blade cartridges for use with a razor handle. The ratio of single-use products to a disposable razor is preferably from 1:1 to 10:1. And the ratio of singe-use products to blade cartridges is preferably from 1:1 to 15:1.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of Exemplary Embodiments and Features are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A consumer product, comprising:
a) a dispenser comprising a compartment, an applicator comprising a skin-contacting surface, and a seal or valve designed and configured to contain materials within the compartment prior to use; and
b) a single-use volume of a shaving composition disposed within the compartment.

2. The product of claim 1, wherein the seal or valve comprises a frangible seal disposed between the compartment and the applicator.

3. The product of claim 1, wherein the seal or valve comprises a seal removably affixed to at least a portion of the applicator.

4. The product of claim 1, wherein the shaving composition comprises a lubricant selected from the group consisting of lubricious water soluble polymers, water insoluble particles, hydrogel-forming polymers and mixtures thereof.

5. The product of claim 1, wherein the shaving composition comprises a water dispersible surface active agent.

6. The product of claim 1, wherein the single-use volume is from about 0.5 ml to about 30 ml.

7. The product of claim 1, further comprising a personal care composition disposed on and/or in the applicator.

8. A consumer product, comprising:
a) a dispenser comprising a first compartment, a second compartment, and an applicator comprising a skin-contacting surface;
b) a single-use volume of a shaving composition disposed within the first compartment; and
c) a single-use volume of a personal care composition disposed in the second compartment, the personal care composition being different form the shaving composition.

9. The product of claim 8, wherein the applicator is used for applying both the shaving composition and the personal care composition.

10. The product of claim 8, wherein the applicator is associated with the first compartment and wherein the dispenser comprises a second applicator that is associated with the second compartment.

11. The product of claim 8, wherein the personal care composition is selected from the group consisting of body washes, moisturizers, shampoos, hair conditioners, hair styling compositions, antiperspirant compositions, deodorant compositions, body sprays, aftershave compositions, and toothpastes.

12. The product of claim 8, wherein the personal care composition comprises an antiperspirant and/or deodorant composition, and wherein the product is marketed to men.

13. The product of claim 8, wherein the personal care composition comprises an aftershave composition, and wherein the product is marketed to men.

14. The product of claim 8, wherein the personal care composition comprises a body wash, and wherein the product is marketed to women.

15. The product of claim 8, wherein the personal care composition comprises a shampoo or hair conditioner, and wherein the product is marketed to women.

16. A consumer product, comprising:
a) a dispenser comprising a first compartment, a second compartment, and an applicator comprising a skin-contacting surface;
b) a first volume of a first shaving composition disposed within the first compartment, the first volume being sufficient to shave a first portion of a user's body; and
c) a second volume of a second shaving composition disposed in the second compartment, the second volume being sufficient to shave a second portion of a user's body.

17. The product of claim 16, wherein the first shaving composition and the second shaving composition comprise substantially identical formulations.

18. The product of claim 16, wherein the first shaving composition comprises a different formulation than the second shaving composition.

19. A consumer product, comprising:
a) a dispenser comprising a first compartment, a second compartment, and an applicator comprising a skin-contacting surface; and
b) a single-use volume of a self-heating shaving composition disposed partly within the first compartment and partly within the second compartment, the shaving composition within the first compartment comprising a first shave gel base and an oxidizing agent, and the shaving composition within the second compartment comprising a second shave gel base and a reducing agent.

20. The product of claim 19, wherein the dispenser further comprises a frangible seal disposed between the first compartment and the second compartment to permit pre-mixing of the shaving compositions contained therein.

21. The product of claim 19, wherein the shaving composition within the first compartment and the shaving composition within the second compartment are not pre-mixed before engaging the applicator.

22. A shaving-related kit, comprising:
a) a plurality of replacement blade cartridges for use with a razor handle; and
b) a plurality of single-use products, each of which comprises:
i) a dispenser including a compartment and an applicator comprising a skin-contacting surface; and
ii) a single-use volume of a personal care composition disposed within the compartment.

23. The kit of claim 22, wherein at least some of the plurality of single-use products comprise a shaving composition disposed within the individual dispenser compartments.

24. The kit of claim 22, wherein all of the plurality of single-use products comprise a shaving composition disposed within the individual dispenser compartments.

25. The kit of claim 22, wherein a first portion of the plurality of single-use products comprise a shaving composition disposed within their respective dispenser compartments, and a second portion of the plurality of single-use products comprise an aftershave composition disposed within their respective dispenser compartments.
